SEQUENCE LISTING (27 sequences)

SEQ ID NO: 1 - protein, 127 aa, Homo sapiens
  Transthyretin isoform
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 127 aa, Homo sapiens
  Transthyretin isoform
GPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 127 aa, Homo sapiens
  Transthyretin isoform
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVITNPKE

SEQ ID NO: 4 - protein, 127 aa, Homo sapiens
  Transthyretin isoform
GPTGTGESKCPLMVKVLDAVRGSPAINVAVHVFRKAADDTWEPFASGKTSESGELHGLTAEEEFVEGIYKVEIDTKSYWKALGISPFHEHAEVVFTANDSGPRRYTIAALLSPYSYSTTAVVTNPKE

SEQ ID NO: 5 - protein, 127 aa, Homo sapiens
  Transthyretin isoform
GPTGTGESKCPLMVKVLDAVRGSPAINVAVHVFRKAADDTWEPFASGKTSESGELHGLTTEEEFVEGIYKVEIDTKYYWKALGISPFHEHAEVVFTANDSGPRRYTIAALLSPYSYSTTAVVTNPKE

SEQ ID NO: 6 - protein, 16 aa, Artificial Sequence
  Peptide Variant
DAVRGSPAINVAVHVF

SEQ ID NO: 7 - protein, 16 aa, Artificial Sequence
  Peptide Variant
DAVRGSPAINVAMHVF

SEQ ID NO: 8 - protein, 13 aa, Artificial Sequence
  Peptide Variant
SYSTTAVVTNPKE

SEQ ID NO: 9 - protein, 13 aa, Artificial Sequence
  Peptide Variant
SYSTTAVITNPKE

SEQ ID NO: 10 - protein, 13 aa, Artificial Sequence
  Peptide Variant
GSPAINVAVHVFR

SEQ ID NO: 11 - protein, 13 aa, Artificial Sequence
  Peptide Variant
GSPAINVAMHVFR

SEQ ID NO: 12 - protein, 22 aa, Artificial Sequence
  Peptide Variant
TSESGELHGLTTEEEFVEGIYK

SEQ ID NO: 13 - protein, 22 aa, Artificial Sequence
  Peptide Variant
TSESGELHGLTAEEEFVEGIYK

SEQ ID NO: 14 - protein, 22 aa, Artificial Sequence
  Peptide Variant
YTIAALLSPYSYSTTAVVTNPK

SEQ ID NO: 15 - protein, 22 aa, Artificial Sequence
  Peptide Variant
YTIAALLSPYSYSTTAVITNPK

SEQ ID NO: 16 - protein, 17 aa, Artificial Sequence
  Peptide Variant
IDTKSYWKALGISPFHE

SEQ ID NO: 17 - protein, 17 aa, Artificial Sequence
  Peptide Variant
IDTKYYWKALGISPFHE

SEQ ID NO: 18 - protein, 23 aa, Artificial Sequence
  Peptide Variant
DAVRGSPAINVAVHVFRKAADDT

SEQ ID NO: 19 - protein, 22 aa, Artificial Sequence
  Peptide Variant
TIAALLSPYSYSTTAVVTNPKE

SEQ ID NO: 20 - protein, 23 aa, Artificial Sequence
  Peptide Variant
DAVRGSPAINVAMHVFRKAADDT

SEQ ID NO: 21 - protein, 22 aa, Artificial Sequence
  Peptide Variant
TIAALLSPYSYSTTAVITNPKE

SEQ ID NO: 22 - protein, 16 aa, Artificial Sequence
  Peptide Variant
  VARIANT 13: Xaa is Val or Met
DAVRGSPAINVAXHVF

SEQ ID NO: 23 - protein, 13 aa, Artificial Sequence
  Peptide Variant
  VARIANT 8: Xaa is Val or Ile
SYSTTAVXTNPKE

SEQ ID NO: 24 - protein, 13 aa, Artificial Sequence
  Peptide Variant
  VARIANT 9: Xaa is Val or Met
GSPAINVAXHVFR

SEQ ID NO: 25 - protein, 22 aa, Artificial Sequence
  Peptide Variant
  VARIANT 18: Xaa is Val or Ile
YTIAALLSPYSYSTTAVXTNPK

SEQ ID NO: 26 - protein, 17 aa, Artificial Sequence
  Peptide Variant
  VARIANT 5: Xaa is Ser or Tyr
IDTKXYWKALGISPFHE

SEQ ID NO: 27 - protein, 22 aa, Artificial Sequence
  Peptide Variant
  VARIANT 12: Xaa is Thr or Ala
TSESGELHGLTXEEEFVEGIYK

(19) United States
(12) Patent Application Publication
(10) Pub. No.: US 2020/0088746 A1
(43) Pub. Date: Mar. 19, 2020

(54) QUANTIFICATION OF TRANSTHYRETIN AND ITS ISOFORMS

(71) Applicant: Alnylam Pharmaceuticals, Inc., Cambridge, MA (US)

(72) Inventors: James Butler, Lynnfield, MA (US); Renta Hutabarat, Cambridge, MA (US); Rene Alvarez, Cambridge, MA (US); Bob Xiong, Cambridge, MA (US); Lily Y.T. Li, Cambridge, MA (US)

(21) Appl. No.: 16/394,017

(22) Filed: Apr. 25, 2019

Related U.S. Application Data
(63) Continuation of application No. 15/338,006, filed on Oct. 28, 2016, now abandoned.
Application No. 15/338,006 is a continuation of application No. 14/358,139, filed on May 14, 2014, now abandoned.
(60) Provisional application No. 61/561,328, filed on Nov. 18, 2011.

(51) Int. Cl. G01N 33/78; G01N 33/68; G01N 30/72; C07K 14/575; C12Q 1/37; G01N 33/58

(52) U.S. Cl. CPC G01N 33/78; G01N 33/6848; G01N 30/7233; H01J 49/00; C12Q 1/37; G01N 33/58; C07K 14/575

(57) ABSTRACT

The present invention relates to assays and methods for the detection of transthyretin and its isoforms. Specifically, the assays and methods of the present invention embrace liquid chromatography and mass spectrometry. The present invention also relates to unique peptides and peptide variants useful in the assays and methods.

### CROSS REFERENCE TO RELATED APPLICATIONS

This application is a continuation of U.S. application Ser. No. 15/338,006 filed Oct. 28, 2016, which is a continuation of U.S. application Ser. No. 14/358,139 filed May 14, 2014, entitled, QUANTIFICATION OF TRANSTHYRETIN AND ITS ISOFORMS, which is a 35 U.S.C. § 371 U.S. National Stage Entry of International Application No. PCT/US2012/065407 filed Nov. 16, 2012, entitled, QUANTIFICATION OF TRANSTHYRETIN AND ITS ISOFORMS, which claims the benefit of priority of U.S. Provisional Application 61/561,328 filed Nov. 18, 2011, entitled, QUANTIFICATION OF TRANSTHYRETIN AND ITS ISOFORMS, the contents of each of which are each incorporated herein by reference in their entirety.

### REFERENCE TO SEQUENCE LISTING

The present application is being filed along with a Sequence Listing in electronic format. The Sequence Listing file, entitled 121301_08104_SL.txt was created on Apr. 17, 2019 and is 12,256 bytes in size. The information in electronic format of the sequence listing is incorporated herein by reference in its entirety.

### FIELD OF THE INVENTION

The present invention relates to assays and methods for the detection of transthyretin and its isoforms. Specifically, the assays and methods of the present invention embrace liquid chromatography and mass spectrometry. The present invention also relates to unique peptides and peptide variants useful in the assays and methods.

### BACKGROUND OF THE INVENTION

Transthyretin (TTR), also known as prealbumin, is a tetrameric protein found in plasma and cerebrospinal fluid. The major role of transthyretin is to transport thyroid hormones, such as thyroxin-T4, and retinol through the association with the retinol-binding protein. Transthyretin has a number of point mutations resulting in sequence heterogeneity which are associated with pathological conditions that result in extracellular deposition of amyloid fibers in tissues, known as amyloidosis.

The total concentration of aggregation prone species transthyretin will depend on the extent of tetramer disruption via mutation and the total concentration of mutant transthyretin present in serum. Moreover, circulating levels of wild-type and mutant transthyretin represent important biomarkers for the assessment of pharmacodynamic activity of candidate drugs in clinical development. Therefore, there is a need for an accurate determination of wild-type and mutant transthyretin concentrations in normal and diseased populations in order to improve drug development and clinical outcomes. The present invention embraces such a method.

### SUMMARY OF THE INVENTION

The present invention provides an assay using liquid chromatography followed by mass spectrometry for the detection of transthyretin in a subject. The present invention further provides a method for quantifying both wild-type transthyretin and isoforms of transthyretin in a subject. The present invention also provides kits for using the assay on a sample from a subject for the quantification of transthyretin. The present invention further provides peptides used in the assay of the present invention

In one embodiment, the present invention provides a method for determining the concentration of an isoform of transthyretin is a sample obtained from a subject. In this method, the sample is treated to digest one or more isoforms of transthyretin contained in the sample, a mass spectrometry profile of the digested sample, and the profile is compared to a standard curve created from the analysis of at least one calibration standard selected from the group consisting of SEQ ID NO. 1, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, and variants thereof. The calibration standards may also be used as internal standards. Calculation of the concentration of one or more isoforms of transthyretin contained in the sample is then possible. The digested sample may be subjected to liquid chromatography prior to the generation of the mass spectrometry profile

In some instances, the sample obtained from a subject may be spiked with a known concentration of one or more peptides selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21 and variants thereof. Peptides and/or proteins used to spike the sample may be used as internal standards or as calibration standards. The peptides disclosed herein may comprise a detectable label. The detectable label may be any number of detectable labels in the art. They may be fluorescent or luminescent or radiolabels. Radiolabeled amino acids may comprise those having or more nitrogen-15, carbon-13 or deuterium.

In one embodiment, the isoforms of transthyretin may include SEQ ID NO. 1, SEQ ID NO. 2, SEQ ID NO. 3, SEQ ID NO. 4, or SEQ ID NO. 5

In one embodiment, the sample obtained from a subject may be diluted by a dilution factor from about 20 to about 30.

In one embodiment, the sample may be obtained from a subject who is a patient.

In one embodiment, the sample obtained may be a serum sample. An SDS-PAGE may be performed on the serum sample prior to the sample being treated to digest the one or more isoforms of transthyretin proteins contained therein. In one embodiment, the serum sample obtained from the subject may be treated to deplete at least one serum protein before treating the sample to digest proteins of the isoforms of transthyretin contained therein. The serum proteins depleted in the sample may include albumin and Immunoglobulin G.

In one embodiment, the sample may be obtained from a subject prior to the subject being administered any substance that would alter the expression of an isoform of transthyretin. Altered isoforms of transthyretin may include SEQ ID NO. 1, SEQ ID NO. 2, SEQ ID NO. 3, SEQ ID NO. 4, or SEQ ID NO. 5.

In one embodiment, a standard curve may be generated using at least 6 or more data points within the range of about 5 ng/mL to about 2500 ng/mL peptide concentration. The standard curve may have a lower data point at 5 ng/mL peptide concentration and an upper data point at 2500 ng/mL peptide concentration. Between the upper and lower data point the standard curve may also have at least 4 data points.

In one embodiment, the standard curve has a maximum bias of no more than 20%.

In one embodiment, digestion of the sample obtained from the subject may be performed using trypsin, endoproteinase Glu-C and/or chymotrypsin.

In one embodiment, the mass spectrometry profile may be generated by performing liquid chromatography followed by tandem mass spectrometry.

In one embodiment, a kit may be provided to quantify the level of one or more isoforms of transthyretin in a sample. The kit may comprise two or more calibration standards selected from the group consisting of SEQ ID NO. 1, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17 and variants thereof.

In one embodiment, a synthetic isolated peptide 10 to 25 amino acids in length having at least 8 contiguous amino acids of a peptide selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, and SEQ ID NO. 21 is provided.

In one embodiment, a synthetic isolated peptide 13 to 18 amino acids in length having the sequence DAVRGSPAINVAXHVF (SEQ ID NO. 22) is provided, where X may be an amino acid having a hydrophobic side chain and the amino acid having a hydrophobic side chain is selected from the group consisting of alanine, valine, isoleucine, leucine, and methionine.

In one embodiment, a synthetic isolated peptide 10 to 15 amino acids in length having the sequence SYSTTAVXTNPKE (SEQ ID NO. 23) or GSPAINVAXHVFR (SEQ ID NO. 24) is provided, where X may be an amino acid having a hydrophobic side chain and the amino acid having a hydrophobic side chain is selected from the group consisting of alanine, valine, isoleucine, leucine, and methionine.

In one embodiment, a synthetic isolated peptide 20 to 25 amino acids in length having the sequence TSESGELHGLTXEEEFVEGIYK (SEQ ID NO. 27) is provided, wherein X may be an amino acid is threonine or alanine.

In one embodiment, a synthetic isolated peptide 20 to 25 amino acids in length having the sequence YTIAALLSPYSYSTTAVXTNPK (SEQ ID NO. 25) is provided, where X may be an amino acid having a hydrophobic side chain and the amino acid having a hydrophobic side chain is selected from the group consisting of alanine, valine, isoleucine, leucine, and methionine.

In one embodiment, a synthetic isolated peptide 14 to 19 amino acids in length having the sequence IDTKXYWKALGISPFHE (SEQ ID NO. 26) is provided, where X may be an amino acid having a polar uncharged side chain and the amino acid having a polar uncharged side chain is selected from the group consisting of serine, threonine, asparagines, glutamine, histidine, tyrosine, cysteine and tyrosine.

In one embodiment, the peptide may be from 13 to 22 amino acids in length. The peptide may further comprise at least one detectable label. The detectable label may be any number of detectable labels in the art. They may be fluorescent or luminescent or radiolabels. Radiolabeled amino acids may comprise those having or more nitrogen-15, carbon-13 or deuterium.

The detectable label may be located on an amino acid selected from the group consisting of valine, phenylalanine and proline. In one embodiment, the detectable label deuterium may be located on the alpha carbon and/or side chain carbons of valine.

In one embodiment, the detectable label deuterium may be located on the alpha carbons and/or side chain carbons of valine and phenylalanine.

In one embodiment, the detectable label deuterium may be located on the alpha caron and/or side chain carbon of valine and the detectable label carbon-13 or nitrogen-15 may be located on proline. In this instance, if the detectable label is carbon-13 it may be located on the alpha carbon and/or side chain carbons of proline; if the detectable label is nitrogen-15 it may be located on the nitrogen of proline.

### DETAILED DESCRIPTION OF THE INVENTION

The present invention provides tandem mass spectrometry assays to be used with liquid chromatography. The present invention also provides for kits and peptides useful in the methods and assays of the present invention. These assays are useful in research and for the detection, diagnosis, prognosis and treatment of certain types of transthyretin (TTR) related pathological conditions, in particular, transthyretin-mediated amyloidosis (ATTR)

Transthyretin is a tetrameric protein found in plasma and cerebrospinal fluid. It has been studied extensively due to the great number of point mutations that result in sequence heterogeneity. Many of these variants are associated with pathological conditions that result in the extracellular deposition of amyloid fibers in tissues such as ATTR. ATTR is associated with the extracellular deposition of wild-type (wt) transthyretin and its mutants as amyloid fibrils in various tissues and organs. A definitive diagnosis of ATTR depends on the detection and identification of transthyretin variants in the body.

The present invention provides a new method for quantifying isoforms of transthyretin, including wild-type and mutant transthyretin, in sample from a subject as well as novel peptide signatures and standards that are useful for this new method

According to the present invention, the concentration of an isoform of transthyretin contained in a sample obtained from a subject may be determined by treating the sample from the subject to digest one or more isoforms of transthyretin proteins contained in the sample. After digestion the sample may be analyzed by mass spectrometry to generate a mass spectrometry profile. The mass spectrometry profile of the digested sample is then be compared to a standard curve to calculate the concentration of one or more isoforms of transthyretin contained in the sample. Liquid chromatography may also be used in the method of analyzing the sample prior to generating the mass spectrometry profile

As used herein, the term “transthyretin,” (TTR) refers to a protein carrier of the thyroid hormone thyroxine and retinol. The term includes truncated transthyretin, fragments of transthyretin, mutated transthyretin, modified transthyretin or isoforms of transthyretin. As used herein, “truncated” refers to a protein or peptide which has been cut short. The term “fragment” relates to an isolated or incomplete portion of a protein or peptide. As used herein, “mutated” refers to a protein or peptide which has undergone a change or alteration in at least one of its amino acids. The term “modified” relates to a protein or peptide which has undergone a chemical and/or a structural alteration. The term “isoform” relates to two or more functionally similar proteins or peptides having a similar but not identical amino acid sequence. A modification of transthyretin can be due to enzymatic or chemical modification. The term transthyretin is also used to designate monomeric or multimeric forms of transthyretin. Transthyretin may also be designated prealbumin. As used herein, “isoforms of transthyretin” include natural and synthetic forms of transthyretin. Natural forms of transthyretin include the naturally occurring, native, variants of transthyretin found in nature such as, but not limited to, the wild-type variant. Synthetic transthyretin includes any form of transthyretin which have been chemically synthesized.

As used herein, the term “variant” refers to proteins with some differences in their amino acid sequences as compared to a natural form of transthyretin. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence. A variant includes substitutional variants, insertional variants, and deletional variants.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Substitutional variants,” when referring to proteins, are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

Such modifications are within the ordinary skill in the art and are performed without undue experimentation.

In some instances, the concentration of transthyretin determined in the sample obtained from a subject may be selected from one or more isoforms of transthyretin. The isoforms of transthyretin may be selected from SEQ ID NO. 1, SEQ ID NO. 2, SEQ ID NO. 3, SEQ ID NO. 4, and SEQ ID NO. 5. SEQ ID NO. 1 is amino acids 21 through 147 of wild-type transthyretin as listed in Genbank (NM_000371) where the signal peptide (amino acids 1-20) are not shown. SEQ ID NO. 2, SEQ ID NO. 3, SEQ ID NO. 4, and SEQ ID NO. 5 are substitutional variants of SEQ ID NO. 1 having a single amino acid substitution in the sequence. SEQ ID NO. 2 is a substitutional variant known as V30M where there is a single amino acid substitution of methionine for valine at position 30 of the wild-type transthyretin sequence (SEQ ID NO. 1). SEQ ID NO. 3 is a substitutional variant known as V122I where there is a single amino acid substitution of isoleucine for valine at position 122 of the wild-type transthyretin (SEQ ID NO. 1). SEQ ID NO. 4 is a substitutional variant, known as T60A, where there is with a single amino acid substitution of alanine for threonine at position 60 of the wild-type transthyretin sequence (SEQ ID NO. 1). SEQ ID NO. 5 is a substitutional variant known as S77Y where there is a single amino acid substitution of tyrosine for serine at position 77 of the wild-type transthyretin sequence (SEQ ID NO. 1).

In the methods of the present invention, a sample may be obtained from a subject. While the sample may include any sample which is amendable for protein analysis, it is most often a serum sample. The sample may be obtained from a subject who is a patient. As used herein, a “subject” refers to a vertebrate, preferably a mammal, more preferably a primate and still more preferably a human.

The sample may be obtained from a subject who is a patient. As used herein, “patient” refers to a subject who may seek or be in need of treatment, requires treatment, is receiving treatment, will receive treatment, or a subject who is under care by a trained professional for a particular disease or condition. The term “treatment” as used herein, means anything which has the effect of ameliorating, reversing, alleviating, inhibiting the progress of, or preventing, either partially or completely, the growth of tumors, tumor metastases, or other transthyretin-related pathological conditions. The term “treating” as used herein, unless otherwise indicated, refers to the act of administering treatment

As used herein, the term “sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecules.

According to the present invention, the sample, once obtained from the subject, may be subjected to enzyme digestion. As used herein, the term “digest” means to break apart into shorter peptides. As used herein, the phrase “treating a sample to digest proteins” means manipulating a sample in such a way as to break down proteins in a sample. In the present invention, one or more isoforms of transthyretin proteins may be digested using enzymes. These enzymes include, but are not limited to, trypsin, endoproteinase Glu-C and chymotrypsin.

The sample obtained from a subject may also be spiked with a known concentration of one or more peptides or proteins. As used herein, the term “spike or spiking” refers to the addition of a known compound. The peptides or proteins used to spike the sample obtained from the subject may be selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, and variants thereof.

Peptides of SEQ ID NO. 6, SEQ ID NO. 8, SEQ ID NO. 10, SEQ ID NO. 12, SEQ ID NO. 14, SEQ ID NO. 16, SEQ ID NO. 18 and SEQ ID NO. 19 are portions of wild-type transthyretin (SEQ ID NO. 1). SEQ ID NO. 6 represents amino acids 18 through 22, SEQ ID NO. 8 represents amino acids 115 through 127, SEQ ID NO. 10 represents amino acids 22 through 34, SEQ ID NO. 12 represents amino acids 49 through 70, SEQ ID NO. 14 represents amino acids 105 through 126, SEQ ID NO. 16 represents amino acids 73 through 89, SEQ ID NO 18 represents amino acids 18 through 40 and SEQ ID NO 19 represents amino acids 106 through 127 of the wild-type transthyretin sequence as listed in Genbank (NM_000371).

Peptides of SEQ ID NO. 7, SEQ ID NO. 11 and SEQ ID NO. 20 are portions of the V30M substitutional variant of wild-type transthyretin protein (SEQ ID NO. 2). SEQ ID NO. 7 represents amino acids 18 through 33, SEQ ID NO. 11 represents amino acids 22 though 34 and SEQ ID NO. 20 represents amino acids 18 through 40 of V30M. The peptides of SEQ ID NO. 9, SEQ ID NO. 15 and SEQ ID NO. 21 are portions of the V122I substitutional variant of wild-type transthyretin protein (SEQ ID NO. 3). SEQ ID NO. 9 represents amino acids 115 through 127, SEQ ID NO. 15 represents amino acids 105 through 126 and SEQ ID NO. 21 represents amino acids 106 through 127 of V122I. SEQ ID NO. 13 represents amino acids 49 through 70 of the T60A substitutional variant of wild-type transthyretin protein (SEQ ID NO. 4). SEQ ID NO. 17 represents amino acids 73 through 89 of the S77Y substitutional variant of wild-type transthyretin (SEQ ID NO. 5).

The peptides used may also be variants of the wild-type transthyretin protein (SEQ ID NO. 1). SEQ ID NO. 22, SEQ ID NO. 23, SEQ ID NO. 24 and SEQ ID NO. 25 are variant peptides of SEQ ID NO. 1 where X may be an amino acid having a hydrophobic side chain. The amino acid may be selected from the group of alanine, valine, isoleucine, leucine, and methionine. SEQ ID NO. 26 is a variant peptide of SEQ ID NO. 1 where X may be an amino acid having a polar uncharged side chain. The amino acid may be selected from serine, threonine, asparagines, glutamine, histidine, tyrosine, cysteine, and tyrosine. SEQ ID NO. 27 is a variant peptide of SEQ ID NO. 1 where X may be threonine or alanine

The peptides or proteins of the present invention spiked into the sample may also comprise at least one detectable label. The detectable label may be selected from nitrogen-15, deuterium, and carbon-13. In some instances, the spike may be used as an internal standard. As used herein, “internal standard” refers to a chemical substance that is added in a constant amount to samples, blanks and calibration standards.

The sample obtained from a subject may also be diluted by a dilution factor. As used herein, “diluted” refers to making the sample weaker in force, content or value by modifying the sample or adding other elements to it. The term “dilution factor” refers to the ratio of the final volume of the diluted sample divided by the initial volume of the sample. The sample may be diluted before the sample is treated to digest the proteins of the isoforms of transthyretin contained therein or after the digestion of the proteins within the sample has occurred. The sample may be diluted by a dilution factor from a range of about 20 to about 30.

In one instance, the sample may obtained from a subject prior to the subject being administered a substance that may alter the expression of at least one isoform of transthyretin. As used here, the phrase “alter the expression” refers to changing or to cause a change in the character or composition of an expression. The substance may alter the expression of an isoform of transthyretin selected from SEQ ID NO. 1, SEQ ID NO. 2, SEQ ID NO. 3, SEQ ID NO. 4 and SEQ ID NO. 5. The sample may also be obtained from the subject after the subject had been administered a substance that altered the expression of at least one isoform of transthyretin. The altered transthyretin isoforms may be selected from the group of SEQ ID NO. 1, SEQ ID NO. 2, SEQ ID NO. 3, SEQ ID NO. 4 and SEQ ID NO. 5

The serum sample may also be subjected to SDS-PAGE. As used herein, “SDS-PAGE” refers to sodium dodecyl sulfate polyacrylamide gel electrophoresis which separates proteins according to their electrophoretic mobility. SDS-PAGE may be performed on the serum sample prior to the sample being treated for digestion of the one or more isoforms of transthyretin contained in the sample. Any transthyretin band may then be excised before the sample is treated for the digestion of one or more isoforms of transthyretin proteins contained therein.

The serum sample obtained from the subject may also be treated to deplete at least one serum protein contained in the sample. The term “serum protein” refers to any protein found in serum. As used herein, the term “deplete” refers to lessening in concentration of a substance in a sample. The substance may be minimally reduced in the sample or it can be completely removed from the sample. In certain instances, it is advantageous to minimize the amount of non-transthyretin proteins contained in the sample. These include, but are not limited to, albumin and Immunoglobulin G.

The sample obtained from a subject may be a serum sample. The sample may be loaded on a polyclonal transthyretin antibody column in order to enrich transthyretin contained within the sample. The serum sample may also be loaded on polyclonal transthyretin antibody beads in order to enrich transthyretin contained in the serum sample. The enriched sample may then be treated to digest one or more isoforms of transthyretin which may be contained in the sample before the sample may be analyzed. The digested sample may be analyzed by liquid chromatography followed by mass spectrometry

The present invention provides a synthetic isolated peptide 10 to 25 amino acids in length having at least 8 contiguous amino acids of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, and SEQ ID NO. 21. In some instances, the peptide of the present invention is from 13 to 22 amino acids in length.

As used herein, “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids and the polypeptide is termed a peptide. If the protein is a peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex such as a dimer, trimer or tetramer. The term protein may also apply to amino acid polymers in which one or more amino acid residues are an artificial chemical analogue of a corresponding naturally occurring amino acid.

Any peptide or protein of the present invention may contain one or more detectable labels. They may be partially labeled or completely labeled throughout. As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, strepavidin and haptens, quantum dots, and the like. Detectable labels may be located at any position in the peptides or proteins disclosed herein. They may be within the amino acids, the peptides, or proteins, or located at the N- or C-termini. Further they may be on the alpha carbon or side chains of the amino acids. In some instances, the detectable label is selected from the radioisotopes nitrogen-15, carbon-13 and deuterium. As used herein, “nitrogen-15” refers to a stable, non-radioactive isotope of nitrogen having 7 protons and 8 neutrons. Nitrogen-15 may also be referred to by the term “heavy nitrogen.” The term “carbon-13” refers to a natural, stable isotope of carbon having 7 neutrons and 6 protons. The term “deuterium” (D) refers to an isotope of hydrogen that has one proton and one neutron in its nucleus and has twice the mass of ordinary hydrogen. Deuterium may also be referred to as heavy hydrogen.

In some embodiments, a detectable label may be located on at least one an amino acid selected from the group consisting of valine, phenylalanine and proline. In some instances the detectable label deuterium may be located on the alpha carbon and/or side chain carbons of the amino acid valine or phenylalanine. As used herein, “alpha carbon” refers to the first carbon preceding carboxylic acid in a peptide bond. The term “side chain carbon” refers to any carbon in the side chain (or R group) which is attached to the alpha carbon.

In some instances the detectable label is located on the amino acid valine and one other amino acid selected from phenylalanine and proline. If the detectable label is deuterium it may be located on the alpha carbons and/or the side chain carbons of the amino acids valine and phenylalanine. If the detectable label is carbon-13 and/or nitrogen-15, the detectable may be located on proline. The detectable label carbon-13 may be attached to the alpha carbon and/or side chain carbons of proline and the detectable label nitrogen-15 is located on the nitrogen of proline. The detectable label nitrogen-15 may also be used to label a peptide where the detectable label may be located on each of the nitrogen within of the peptide

In some embodiments, peptides or proteins of the invention may be isotopically enriched or altered. The isotope may comprise a hydrogen isotope such as deuterium or tritium or any other isotope of an element found in the peptide or protein

In some instances, a peptide may be 10 to 25 amino acids in length having a sequence selected from the group consisting of SEQ ID NO. 22, SEQ ID NO. 23, SEQ ID NO. 24, and SEQ ID NO. 25, where X is defined as an amino acid having a hydrophobic side chain. The hydrophobic side chain may be selected from the group consisting of alanine, valine, isoleucine, leucine, and methionine. The peptide having the SEQ ID NO. 22 may be 13 to 18 amino acids in length and X is an amino acid selected from valine or isoleucine and the peptides having the SEQ ID NO. 23 and SEQ ID NO. 24 may be 10 to 15 amino acids in length. The amino acid having a hydrophobic side chain in SEQ ID NO. 23 may be selected from valine or isoleucine and in SEQ ID NO. 24 the amino acid may be selected from valine or methionine. The peptide having the SEQ ID NO. 25 may be 20 to 25 amino acids in length and X may be an amino acid selected from valine or isoleucine

The peptide may also be 20 to 25 amino acids in length having a sequence SEQ ID NO. 27, where X may be an amino acid selected from threonine or alanine. In one instance, a peptide may be 14 to 19 amino acids in length having a sequence SEQ ID NO. 26, where X may be an amino acid having a polar uncharged side chain. The polar side chain may be selected from the group consisting of serine, threonine, asparagines, glutamine, histidine, tyrosine, cysteine and tyrosine.

To generate the mass spectrometry profile liquid chromatography is performed followed by tandem mass spectrometry (LC/MS/MS). As used herein “tandem” refers to an arrangement of two or more objects, processes, methods, procedures or steps placed substantially end to end

As used herein, the term “liquid chromatography” (LC) means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., the mobile phase), as this fluid moves relative to the stationary phase(s). LC includes reverse phase liquid chromatography (RPLC), high performance liquid chromatography (HPLC) and high turbulence liquid chromatography (HTLC)

The term “mass spectrometry profile” refers to the output generated from analysis of a sample by a mass spectrometer. The term “mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” (MS) refers to the use of a mass spectrometer to detect gas phase ions. The output generated by the mass spectrometer may be represented by a mass spectrum, a mass chromatograph, or a three-dimensional contour map where the mass-to-charge ratio is on the x-axis, intensity is on the y-axis and an additional parameter such as time is on the z-axis. An output as a mass spectrum is an intensity versus mass-to-charge ratio plot that represents the chemical analysis. A mass chromatograph is a representation of mass spectrometry data in an intensity versus time chromatogram. Types of mass chromatographs include selected ion monitoring (SIM), total ion current (TIC), and selected reaction monitoring chromatogram (SRM).

The standard curve used in the present invention can be created using at least one calibration standard. The calibration standards used to generate the calibration curve may be selected from the group consisting of SEQ ID NO. 1, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17 and variants thereof. As used herein, the term “calibration standard” refers to a sample containing a known concentration of an analyte. The phrase “standard curve” refers to a curve created by analyzing at least 2 calibration standards using the same methods as the analysis of the sample.

The standard curve can be generated using at least 2, at least 3, at least 4, at least 5, at least 6, at least 7 or at least 8 data points within the range of about 5 to about 2500 ng/mL of the peptide concentration in the calibration standard. In one instance, the standard curve is generated using at least 6 data points. The standard curve may also have a lower data point at about 5 ng/mL, an upper data point at 2500 ng/mL and at least 4 data points in the range of about 5 ng/mL to about 2500 ng/mL of peptide concentration. The standard curve created in the present invention may also have a maximum bias of no more than 20%. As used herein, “bias” refers to the percentage calculated from the equation [(mean calculated concentration−expected concentration)/expected concentration]×100

The concentration of an isoform of transthyretin in a sample may be determined before a subject has received treatment for a transthyretin-related pathological condition. The concentration of an isoform of transthyretin may also be determined in a subject after the subject has received treatment for a transthyretin-related pathological condition. The concentration of the isoform of transthyretin in the subject before treatment and after treatment can then be compared to determine the impact treatment may be having on the transthyretin-related pathological condition.

In one instance, a kit may be used to quantify the level of one or more isoforms of transthyretin contained in a sample. The kit may comprise two or more calibration standards selected from the group of SEQ ID NO. 1, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, and SEQ ID NO. 17 and variants thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Preparation of Standard Curves

A standard curve of analyte concentration versus analyte peak area ratio (analyte/internal standard) was generated for each isoform of transthyretin using liquid chromatography followed by tandem mass spectrometry (LC/MS/MS). Stable-labeled peptides, listed in Table 1, were used to generate a standard curve over the concentration range 5 ng/mL to 2500 ng/mL. Concentrations of 2500, 1000, 500, 250, 100, 50, 20, 10 and 5 ng/mL were selected to generate the standard curve for the stable-labeled peptides. Each concentration was run through the LC/MS/MS twice to create a set of data points for each concentration. Linear regression (weighing 1/x²) was used to process the data points from the LC/MS/MS analysis to generate the standard curve.

TABLE 1
Peptides Used to Generate Standard Curves

| Peptide | SEQ ID NO. |
|---|---|
| DAVRGSPAINVAVHVF | 6 |
| DAVRGSPAINVAMHVF | 7 |
| SYSTTAVVTNPKE | 8 |
| SYSTTAVITNPKE | 9 |

### Example 2. Digestion of Proteins in the Sample

Before a sample was evaluated for transthyretin isoform concentration, digestion of the proteins in the sample was performed. Pooled serum was first diluted with MilliQ water to produce a 25-fold dilution. The dilute serum was then denatured in a heat block at 100° C. for 10 minutes and then quenched on ice immediately for 10 minutes following heat denaturation. To collect condensation on the sides of the sample tubes, the tubes were spun briefly before dithiothreitol (DTT) was added to each tube at the top of the liquid level for reduction. After slight agitation by hand, the sample was incubated at room temperature for 30 minutes before Iodoacetamide was added to each tube and incubated in the dark at room temperature for 30 minutes (alkylation). The samples were then denatured again in a heat block at 100° C. for 10 minutes and quenched on ice immediately for 10 minutes following heat denaturation. Three reagents were added in the order of 15 μL of 1M tris-hyrdochloride (Tris-HCl), 8 μL of 200 mM Ca₂Cl and 5 μL of 1 μg/μL chymotrypsin to the denatured sample before the sample was incubated for three hours at 30° C. After incubation the samples were precipitated with 240 μL of acetonitrile, vortex-mixed and then centrifuged at 12,000 rpm for ten minutes. Two aliquots of 175 μL each were drawn from the supernatant and transferred to two deep well plates before being dried under a steady stream of nitrogen.

### Example 3. LC/MS/MS Method

After a sample has been treated to digest the proteins of the isoforms of transthyretin contained in the sample, the sample is extracted with 240 μL of acetonitrile. Two aliquots (175 μL) of the supernatant are dried down in deep well plates under a steady stream of nitrogen. One plate is reconstituted in 50 μL of mobile phase A (95% water to 5% acetonitrile by volume with 0.1% formic acid) for analysis using LC-MS/MS. The second plate is stored at −80° C. as backup.

The one aliquot of digested sample was analyzed first by liquid chromatography using a Varian Metasil C18 2×50 mm column heated at 40° C. 20 μL of the digested sample was pumped through the column with mobile phase B (50% methanol and 50% acetonitrile by volume with 0.1% formic acid) and mobile phase A at a flow rate of 0.6 mL/min at the gradient listed in Table 2. The analysis of the digested sample by liquid chromatography began at 0.01 minutes and ended at 4.50 minutes. The sample needle was washed with a solution of 75% water to 25% acetonitrile with 1% formic acid before the next sample was analyzed.

TABLE 2
Mobile Phase B Gradient

| Time (min.) | Percentage of Mobile Phase B |
|---|---|
| 0.01 | 0 |
| 3.40 | 40 |
| 3.50 | 90 |
| 4.00 | 90 |
| 4.10 | 0 |
| 4.50 | 0 |

After the aliquot of digested sample was analyzed by liquid chromatography, the sample was then analyzed by tandem mass spectrometry. The mass spectrometer used to analyze the digested sample was the API 5000 LC/MS/MS system by AB SCIEX of Foster City, Calif. The ion source was a turbo ion spray at a voltage of 5000V with a declustering potential at 60V. The polarity of the tandem mass spectrometry was in positive mode and the source temperature was 500° C. The mass spectrometry data of the digested sample used to quantify isoforms of transthyretin in the sample were acquired by multiple reaction monitoring (MRM).

### Example 4. Sample Quantification

A serum sample obtained from a subject was treated to digest the isoforms of transthyretin before the sample was run on LC/MS/MS. Peak area ratios (analyte/internal standard) were used to calculate the concentrations of the anaytes against standard curves established for each analyte. A mass spectrometry profile was generated by the tandem mass spectrometer displaying peaks on a time versus intensity graph. The peak areas of the isoform of transthyretin contained in the sample was determined by calculating the area under the curve. The sample peak areas were then divided by the internal reference standard peak areas for the same isoform of transthyretin in order to calculate a peak area ratio. The peak area ratio was then compared to the standard curve representing the isoform of transthyretin contained in the sample to calculate the actual concentration of the isoform in the sample.

### Example 5. Assessment of Treatment Effects

The present invention may be used to determine whether a therapeutic treatment alters the expression of any transthyretin isoform. A sample is obtained from a patient before the patient is administered a substance which alters the expression of at least one isoform of transthyretin. A sample is then obtained from the same patient after the administration of the substance altering the expression of at least one isoform of transthyretin. Both samples are then run through the LC/MS/MS to determine the concentrations of the isoforms of transthyretin contained in the samples. The concentrations of the isoforms of transthyretin were calculated based on their corresponding standard curves using peak area ratios of the monitored peptides A comparison of the concentrations of the isoforms contained in the two samples will be able to show if the administration of the substance changed the concentration of the isoforms of transthyretin in the patient.

### Example 6. Nitrogen-15 Labeling of Peptides and Proteins

### Forming an Unlabeled Bacteria Suspension

LB and kanamycin (50 μg/ml) agar plates were streaked with E. coli (strain: BL21(DE3)) freezer stocks containing a pet41 plasmid expressing Met-hTTR (human transthyretin with an N-terminal methionine residue added) and incubated overnight at 37° C. About 50 ml of LB and 50 μg/ml of Kanamycin were inoculated with bacteria from the agar plate and incubated on a shaker at 37° C. and 220 rpm. When the optical density at 600 nm (OD600) reached about 0.1-0.5 1.0 L of the LB and kanamycin (50 μg/ml) culture were inoculated with approximately 15 ml of starter culture and incubated on the shaker at 37° C. and 220 rpm. When OD of the 1.0 L culture was approximately 0.6, 0.35 ml 1M isopropyl-1-thio-β-D-galactopyranoside (IPTG), which had been passed through a 0.2 μm filter, was added to the culture and incubated on a shaker at 20° C. and 220 rpm overnight.

After 16-20 hours of induction, the cells were then harvested by centrifugation at 8K×g (8000 times gravity) for 10 minutes to form a bacterial suspension.

### Forming a Labeled Bacteria Suspension

LB and kanamycin (50 μg/ml) agar plates were streaked with E. coli (strain: BL21(DE3)) freezer stocks containing a pet41 plasmid expressing Met-hTTR (human transthyretin with an N-terminal methionine residue added) and incubated overnight at 37° C. About 50 ml of minimal media were inoculated with one bacterial colony, using as little material (cells and media) as possible, and then incubated on the shaker overnight at 25° C. and 220 rpm. The overnight cultures were diluted 100× into 1.0 L fresh minimal media, where the culture media was prewarmed before inoculation, and then incubated on the shaker at 37° C. and 220 rpm.

When the culture OD600 reached about 0.6, 0.35 ml 1M isopropyl-1-thio-β-D-galactopyranoside (IPTG), which has been passed through a 0.2 μm filter, was added to the culture and incubated on a shaker at 20° C. and 220 rpm overnight.

After 16-20 hours of induction, the cells were then harvested by centrifugation at 8K x g (8000 times gravity) for 10 minutes to form a bacterial suspension.

### Purification of the Peptides and Proteins

The bacterial suspension was re-suspended in a lysis buffer created from 20 mM TRIS (tris(hydroxymethyl)aminomethane) at a pH of 8.0, 2 mM BME (beta-mercaptoethanol), and lysozyme so there was about 50 ml of buffer per liter of cell culture harvested. The suspension was then frozen at −20° C. and stored at −80° C. The bacterial suspension was thawed at room temperature, and refrozen again on liquid nitrogen. It was re-thawed and incubated at room temperature until the suspension was viscous. Deoxyribonuclease I (DNase I) was first dissolved in 1 ml of 1M magnesium chloride and added to the viscous pellet. The pellet and DNase I was incubated at room temperature for about 1 hour or until the viscosity cleared and before the sample was centrifuged at 16K x g for 30 minutes at 4° C. The sample was then decanted and the supernatant was retained. Ethylenediaminetetraacetic acid (EDTA) was added to the supernatant to reach 0.1 mM and Beta-mercaptoethanol (BME) was then added to reach the final concentration of 2 mM before the supernatant solution was mixed. Solid ammonium sulfate was slowly added, by stirring, to the solution until the solution reached 45% saturation, and then the solution was stirred at room temperature for 3 hours. The supernatant solution was decanted and while the solution was stirred solid ammonium sulfate was added until the solution reached 90% saturation. The 90% saturated solution was stirred at room temperature for 1 hour before the solution was centrifuged at 16K x g for 20 minutes at 4° C.

The centrifuged solution was solubilized in 20 ml phosphate-buffered saline (PBS) before it was dialyzing against 4 L of 5 mM TRIS at a pH of 8.0, 0.1 mM EDTA, 2 mM BME overnight at 4° C. in 10K MWCO (10,000 molecular-weight cutoff) dialysis tubing. A portion of the dialyzed sample was run on a SDS-PAGE gel to assess the purification procedure. After the purification was checked, the dialysates were collected and heated at 60° C. for 30 minutes before they were centrifuged for 15 minutes at 16K x g. The supernatant was then passed through a 0.2 μm filter and loaded onto a QSepharose HP anion exchange column. The filtered supernatant was eluted from the column with an elution buffer (10 mM HEPES (4-(2-hydroxyethyl)-1-piperazineethanesulfonic acid) at a pH of 7.5, 0.1 mM EDTA, 1 mM dithiothreitol (DTT), 1 M sodium chloride) using the following scheme: 0-10% elution buffer over 2 column volumes (cv), where 1 cv is about 55 ml; 10-40% elution buffer over 15 cv; 100% elution buffer for 1 cv; 11 nil fraction size was used for all steps.

The fractions are analyzed by SDS-PAGE and then combined with other fractions of the same purity. The combined fractions were then concentrated using a 10K MWCO centrifugal concentrator at 3.5K x g and 4° C. Solid ammonium sulfate was added to the concentrated fractions to reach about 1.5M before the fractions were loaded onto a Phenyl Sepharose HP hydrophobic interaction column. The column was washed with 2 cv loading buffer (10 mM HEPES (4-(2-hydroxyethyl)-1-piperazineethanesulfonic acid) at a pH of 7.5, 0.1 mM EDTA, 1 mM dithiothreitol (DTT), 1.5 M ammonium sulfate); 8 ml fractions were collected during this step. The concentrated fractions were eluted using the following scheme: 0-40% elution buffer over 2 cv (10 mM HEPES (4-(2-hydroxyethyl)-1-piperazineethanesulfonic acid) at a pH of 7.5, 0.1 mM EDTA, and 1 mM dithiothreitol (DTT)), 8 ml fraction size, where 1 cv is about 20 ml; 40-62% elution buffer over 15 cv, 5 ml fraction size; 100% elution buffer over 5 cv, 5 ml fraction size.

The fractions were analyzed by SDS-PAGE and combined with other fractions of the same purity before being concentrated using a 10K MWCO centrifugal concentrator at 3.5K x g and 4° C. The concentrated fractions were then dialyzing against 4 L of 10 mM HEPES at a pH of 7.5, 0.1 mM EDTA, 2 mM BME overnight at 4° C. in 10K MWCO dialysis tubing. The dialyzed sample was then loaded on a Source 15Q anion exchange column and washed with a buffer of comprising 10 mM HEPES (4-(2-hydroxyethyl)-1-piperazineethanesulfonic acid) at a pH of 7.5, 0.1 mM EDTA, and 1 mM dithiothreitol (DTT). The concentrated fractions were then eluted with elution buffer (10 mM HEPES (4-(2-hydroxyethyl)-1-piperazineethanesulfonic acid) at a pH of 7.5, 0.1 mM EDTA, 1 mM dithiothreitol (DTT), and 1 M sodium chloride) using the following scheme: 0-10% elution buffer over 1 cv, 5 ml fraction size where 1 cv is about 6 ml; 10-30% elution buffer over 30 cv, 3 ml fraction size; 30-100% elution buffer over 3 cv, 5 ml fraction size.

The resulting fractions were then combined based on the relative concentration per the UV absorbance signal of the fractions. The fractions were then concentrated using a 10K MWCO centrifugal concentrator at 3.5K x g and 4° C. so the final volume was less than 10 ml. The concentrated fractions were then loaded on a Superdex 75 26/60 column equilibrated in a buffer of 1×PBS, 0.01 mM EDTA and 1 mM DTT. The samples were eluted using the equilibration buffer, collected in 4 ml fractions and combined based on their relative concentrations based on their UV absorbance signal. The combined fractions were then filtered through a 0.2 μm filter and stored at 4° C. The concentrations of the fractions were measured by UV/VIS absorbance spectroscopy where the extinction coefficient for PBS was 1.41 (mg/ml)⁻¹ cm⁻¹. Once concentration was determined, the samples were aliquotted and stored at −80° C. The purity of the concentrated fractions was assessed by SDS-PAGE and size exclusion chromatography (Superdex 75 10/300 column) using a buffer of 1×PBS. The final sample was submitted for MALDI-TOF mass spectrometry analysis before completing the certificate of analysis.

1. A method of determining the concentration of an isoform of transthyretin in a sample comprising:
(a) obtaining a sample from a subject;
(b) treating said sample to digest one or more isoforms of transthyretin proteins contained therein;
(c) generating a mass spectrometry profile of the digested sample of step (b);
(d) comparing the mass spectrometry profile from step (c) to a standard curve, wherein said standard curve has been created using at least one calibration standard selected from the group consisting of SEQ ID NO. 1, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17 and variants thereof; and
(e) calculating a concentration of said one or more isoforms of transthyretin in the sample obtained from the subject based on the standard curve.

2. The method of claim 1 wherein the sample of step (b) is subjected to liquid chromatography prior to the generation of said mass spectrometry profile.

3. The method of claim 1, further comprising spiking the sample of (a) with a known concentration of one or more peptides or proteins selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, and variants thereof.

4. The method of claim 3, wherein said one or more peptides or proteins comprises a detectable label.

5. The method of claim 4, wherein the detectable label is selected from the group consisting of nitrogen-15, carbon-13 and deuterium.

6. The method of claim 1, wherein said one or more isoforms of transthyretin is selected from the group consisting of SEQ ID NO. 2, SEQ ID NO. 3, SEQ ID NO. 4, and SEQ ID NO. 5.

7. The method of claim 1, further comprising diluting the sample obtained in (a) by a dilution factor from about 20 to about 30.

8. The method of claim 7, where the dilution factor is 25.

9. The method of claim 1, wherein the subject is a patient.

10. The method of claim 1, wherein the sample is serum.

11. The method of claim 10, where a SDS-PAGE is performed prior to digestion in step (b).

12. The method of claim 10, wherein the sample obtained from the subject is treated to deplete at least one serum protein contained therein before digestion.

13. The method of claim 12, wherein the at least one serum protein is albumin and/or Immunoglobulin G.

14. The method of claim 1, wherein the sample is obtained from said subject prior to administration to said subject of any substance that alters the expression of at least one or more isoforms of transthyretin.

15. The method of claim 14, where the one or more isoforms of transthyretin is selected from the group consisting of SEQ ID NO. 2, SEQ ID NO. 3, SEQ ID NO. 4, and SEQ ID NO. 5.

16. The method of claim 1, wherein the standard curve is generated using at least 6 data points within the range of about 5 to about 2500 ng/mL peptide concentration.

17. The method of claim 16, wherein the standard curve has a lower data point at about 5 ng/mL peptide concentration and an upper data point at about 2500 ng/mL peptide concentration.

18. The method of claim 17, wherein the standard curve is generated having a lower data point at about 5 ng/mL peptide concentration, an upper data point at 2500 ng/mL peptide concentration, and at least 4 data points in the range of about 5 ng/mL to about 2500 ng/mL peptide concentration.

19. The method of claim 1, wherein the standard curve has a maximum bias of no more than 20%.

20. The method of claim 1, where digestion is performed using trypsin.

21. The method of claim 1, where digestion is performed using endoproteinase Glu-C.

22. The method of claim 1, where digestion is performed using chymotrypsin.

23. The method of claim 1, wherein the mass spectrometry is tandem mass spectrometry.
